(19) United States
(12) Patent Application Publication
(10) Pub. No.: US 2013/0198884 A1
(43) Pub. Date: Aug. 1, 2013

(54) SPINACH HYBRID VARIETY NUN 01084

(71) Applicant: Jane Ane Dijkstra, Beegden (NL)

(72) Inventor: Jane Ane Dijkstra, Beegden (NL)

(21) Appl. No.: 13/559,923

(22) Filed: Jul. 27, 2012

(51) Int. Cl. A01H 5/12; C12N 15/82

(52) U.S. Cl. CPC A01H 5/12; C12N 15/8241
USPC 800/260; 800/298; 435/410; 800/278; 435/6.11; 47/58.1 FV

(57) ABSTRACT

The invention provides seed and plants of the spinach variety designated NUN 01084. The invention thus relates to the plants, seeds and tissue cultures of spinach variety NUN 01084, and to methods for producing a spinach plant produced by crossing a plant of spinach variety NUN 01084 with itself or with another spinach plant, such as a plant of another variety. The invention further relates to seeds and plants produced by such crossing. The invention further relates to parts of a plant of spinach variety NUN 01084, including the leaves of such plants.

This application claims priority under 35 U.S.C. 119(a)-(d) to NL-Plant Variety Protection Application No. SPN573, filed by Nunhems B. V. on Dec. 28, 2011, the disclosure of which is herein incorporated by reference in its entirety.

### FIELD OF THE INVENTION

The present invention relates to the field of plant breeding and, more specifically, to the development of spinach hybrid variety NUN 01084 (SCORPIUS).

### BACKGROUND OF THE INVENTION

The goal of vegetable breeding is to combine various desirable traits in a single variety/hybrid. Such desirable traits may include greater yield, resistance to insects or pests, tolerance to heat and drought, better agronomic quality, higher nutritional value, growth rate and fruit properties.

Breeding techniques take advantage of a plant's method of pollination. There are two general methods of pollination: self-pollination and cross-pollination.

Plants that have been self-pollinated and selected for type over many generations become homozygous at almost all gene loci and produce a uniform population of true breeding progeny, a homozygous plant. A cross between two such homozygous plants of different varieties produces a uniform population of hybrid plants that are heterozygous for many gene loci. Conversely, a cross of two plants each heterozygous at a number of loci produces a population of hybrid plants that differ genetically and are not uniform. The resulting non-uniformity makes performance unpredictable.

The development of uniform varieties requires the development of homozygous inbred plants, the crossing of these inbred plants, and the evaluation of the crosses. Pedigree breeding and recurrent selection are examples of breeding methods that have been used to develop inbred plants from breeding populations. Those breeding methods combine the genetic backgrounds from two or more plants or various other broad-based sources into breeding pools from which new lines are developed by selfing and selection of desired phenotypes. The new lines are evaluated to determine which of those have commercial potential.

Spinach (Spinacia oleracea) is a flowering plant in the family Amaranthaceae. Spinach is an annual plant (rarely biennial) having flowers that mature into a small hard dry lumpy fruit cluster about 5-10 mm across containing several seeds.

Spinach has two stages in its life cycle including the vegetative, rosette stage in which the plant is marketable (about 35-40 days) and the bolting, seed stalk stage in which the plant is no longer marketable. Spinach can grow in a range of soils as long as they are moist and fertile, and particularly sandy loams that are high in organic matter.

While breeding efforts to date have provided a number of useful spinach lines with beneficial traits, there remains a great need in the art for new varieties with further improved traits. Such plants would benefit farmers and consumers alike by improving crop yields and/or quality.

### SUMMARY OF THE INVENTION

In one aspect, the present invention provides a seed of NUN 01084 a sample of seed of said variety having been deposited under ATCC Accession Number ______.

Another aspect refers to a plant designated NUN 01084, e.g., grown from the seed a sample of seed of said variety having been deposited under ATCC Accession Number ______ or a plant part thereof such as a seed, pollen, an ovule, a leaf, and a cell.

Also provided is a progeny plant of a plant designated NUN 01084 that comprises all of the physiological and morphological characteristics of NUN 01084, a sample of seed of said variety having been deposited under ATCC Accession Number ______, and seeds thereof.

Also provided is a progeny plant, such as an Essentially Derived Variety, of NUN 01084 having one, two or three physiological and/or morphological characteristics which are different from those of NUN 01084 and which otherwise has all the physiological and morphological characteristics of NUN 01084, wherein a representative sample of seed of variety NUN 01084 has been deposited under Accession Number NCIMB ______

Moreover, the present invention refers to a spinach plant, or a part thereof, having all or essentially all the physiological and morphological characteristics of NUN 01084.

Also provided is a tissue culture of regenerable cells of spinach variety NUN 01084, a sample of seed of said variety having been deposited under ATCC Accession Number ______. In one embodiment, said tissue culture comprises cells or protoplasts from a plant part selected from the group consisting of embryos, meristems, cotyledons, pollen, leaves, anthers, roots, root tips, shoots, pistil, flower, seed and stalks. Also provided is a spinach plant regenerated from said tissue culture, wherein the regenerated plant expresses all or essentially all of the physiological and morphological characteristics of spinach variety NUN 01084, a sample of seed of said variety having been deposited under ATCC Accession Number ______.

Another aspect of the invention refers to a method of producing seed, comprising crossing a plant designated NUN 01084 with itself or a second spinach plant and allowing seed to form. In one embodiment, a plant designated NUN 01084 is crossed with a spinach plant of a different genotype relative to said plant designated NUN 01084. Thus, also provided is an F1 hybrid seed produced by said method and a an F1 hybrid plant produced by growing said F1 hybrid seed.

The invention also refers to a method for producing a seed of a NUN 01084-derived spinach plant comprising the steps of:
(a) crossing a spinach plant designated NUN 01084, a sample of seed of said variety having been deposited under ATCC Accession Number ______, with a second spinach plant; and
(b) allowing seed of a NUN 01084-derived spinach plant to form.

In one embodiment, this method may further comprise the steps of:
(c) crossing a plant grown from said NUN 01084-derived spinach seed with itself or a second spinach plant to yield additional NUN 01084-derived spinach seed;
(d) growing said additional NUN 01084-derived spinach seed of step (c) to yield additional NUN 01084-derived spinach plants; and
(e) repeating the crossing and growing steps of (c) and (d) to generate further NUN 01084-derived spinach plants.

Also provided is a method of vegetatively propagating a plant designated NUN 01084 comprising the steps of:
(a) collecting tissue capable of being propagated from a plant designated NUN 01084, a sample of seed of said variety having been deposited under ATCC Accession Number ______;
(b) cultivating said tissue to obtain proliferated shoots; and
(c) rooting said proliferated shoots to obtain rooted plantlets.

In one embodiment, this method further comprises growing plants from said rooted plantlets.

Further provided is a method of introducing a desired trait into hybrid variety NUN 01084 comprising:
(a) crossing a plant designated NUN 01084, a sample of seed of said variety having been deposited under ATCC Accession Number ______, with a second spinach plant that comprises a desired trait to produce F1 progeny;
(b) selecting an F1 progeny that comprises the desired trait;
(c) crossing the selected F1 progeny with a plant of variety NUN 01084 to produce backcross progeny;
(d) selecting backcross progeny comprising the desired trait and the physiological and morphological characteristic of spinach hybrid variety NUN 01084; and
(e) repeating steps (c) and (d) three or more times in succession to produce selected fourth or higher backcross progeny that comprise the desired trait.

Also provided is a spinach plant produced by this method, wherein the spinach plant comprises essentially all of the physiological and morphological characteristics of spinach hybrid variety NUN 01084, a sample of seed of said variety having been deposited under ATCC Accession Number ______.

Moreover, a method of producing a plant designated NUN 01084, a sample of seed of said variety having been deposited under ATCC Accession Number ______, comprising an added desired trait, the method comprising introducing a transgene conferring the desired trait into a plant of spinach hybrid variety NUN 01084 to produce a spinach plant designated NUN 01084 comprising an added desired trait.

Also provided is a method of determining the genotype of a plant designated NUN 01084, comprising obtaining a sample of nucleic acids from said plant and detecting in said nucleic acids a plurality of polymorphisms.

In one embodiment, this method further comprises the step of storing the results of the step of detecting the plurality of polymorphisms on a computer readable medium.

Moreover, a method of producing spinach comprising:
(a) obtaining a plant designated NUN 01084, and
(b) collecting leaf tissue from the plant.
   is provided herewith.

In still yet another aspect, the invention provides a method of determining the genotype of a plant of spinach variety NUN 01084 comprising detecting in the genome of the plant at least a first polymorphism. The method may, in certain embodiments, comprise detecting a plurality of polymorphisms in the genome of the plant. The method may further comprise storing the results of the step of detecting the plurality of polymorphisms on a computer readable medium. The invention further provides a computer readable medium produced by such a method.

These and other features and advantages of this invention are described in, or are apparent from, the following detailed description of various exemplary embodiments of the devices and methods according to this invention.

### FIGURES

FIG. 1 shows cotyledones of NUN 01084 and PV 0293.

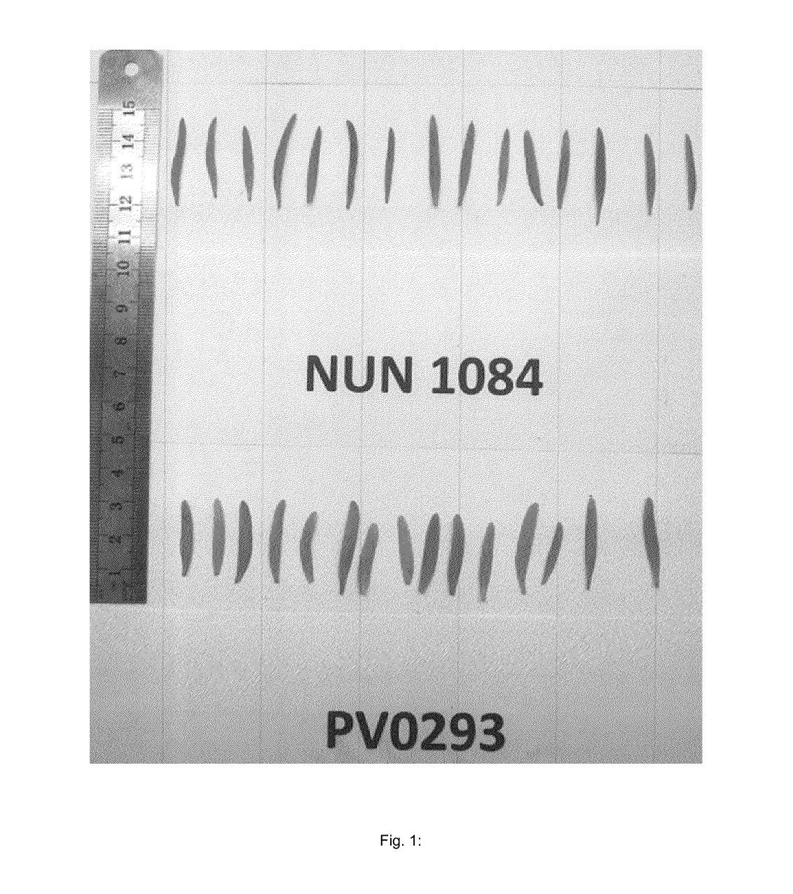

FIG. 2 shows first foliage leaves of NUN 01084 and PV 0293.

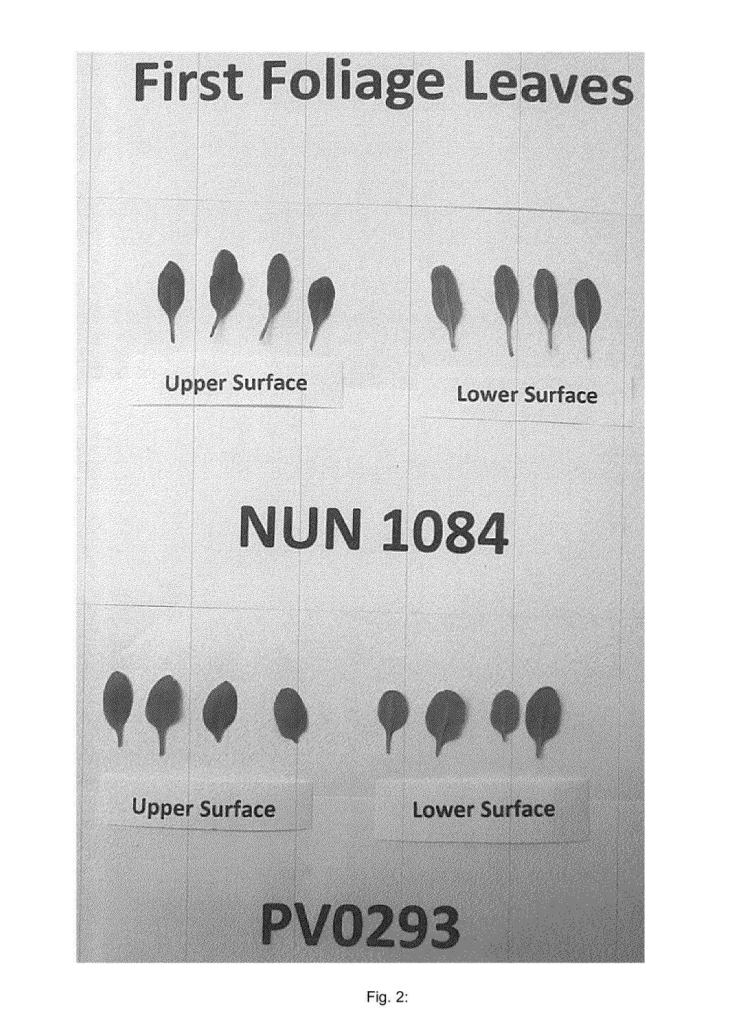

FIG. 3 shows a leave at prime market stage of NUN 01084 and PV 0293.

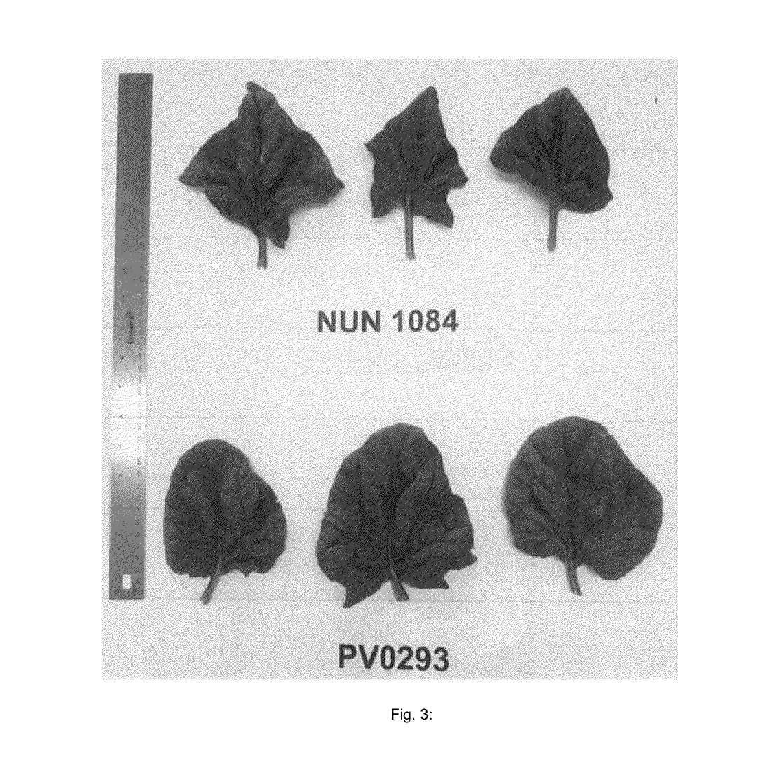

### DEFINITIONS

In the description and tables herein, a number of terms are used. In order to provide a clear and consistent understanding of the specification and claims, the following definitions are provided:

When used in conjunction with the word “comprising” or other open language in the claims, the words “a” and “an” denote “one or more.”

“Spinach” refers herein to plants of the species Spinacia oleracea L.

“Cultivated spinach” refers to plants of Spinacia oleracea L, i.e. varieties, breeding lines or cultivars of the species Spinacia oleracea L, cultivated by humans and having good agronomic characteristics; preferably such plants are not “wild plants”, i.e. plants which generally have much poorer yields and poorer agronomic characteristics than cultivated plants and e.g. grow naturally in wild populations. “Wild plants” include for example ecotypes, PI (Plant Introduction) lines, landraces or wild accessions or wild relatives of a species.

“USDA descriptors” are the plant variety descriptors described for spinach in the “Objective description of Variety Spinach Spinacia oleracea L.)”, ST-470-83 (as published by U.S. Department of Agriculture, Agricultural Marketing Service, Science and Technology, Plant Variety Protection Office, Beltsville, Md. 20705 (available on the world wide web at www.ams.usda.gov/AMSv1.0/) and which can be downloaded from the world wide web at http://www.ams.usda.gov/AMSv1.0/getfile?dDocName=STELDEV3003749.

“UPOV descriptors” are the plant variety descriptors described for spinach in the “Guidelines for the Conduct of Tests for Distinctness, Uniformity and Stability, TG/85/7 (Geneva 2008), as published by UPOV (International Union for the Protection of New Varieties and Plants, available on the world wide web at upov.int) and which can be downloaded from the world wide web at http://www.upov.int/edocs/tgdocs/en/tg055.pdf and is herein incorporated by reference in its entirety.

“RHS” refers to the Royal Horticultural Society of England which publishes an official botanical color chart quantitatively identifying colors according to a defined numbering system, The chart may be purchased from Royal Horticulture Society Enterprise Ltd RHS Garden; Wisley, Woking; Surrey GU236QB, UK, e.g., the RHS colour chart: 2007 (The Royal Horticultural Society, charity No: 222879, PO Box 313 London SW1 P2PE; sold by, e.g., TORSO-VERLAG, Obere Grüben 8•D-97877 Wertheim, Article-No.: Art62-00008 EAN-Nr.: 4250193402112).

As used herein, the term “plant” includes the whole plant or any parts or derivatives thereof, preferably having the same genetic makeup as the plant from which it is obtained, such as plant organs (e.g. harvested or non-harvested leaves), plant cells, plant protoplasts, plant cell and/or tissue cultures from which whole plants can be regenerated, plant calli, plant cell clumps, plant transplants, seedlings, hypocotyl, cotyledon, plant cells that are intact in plants, plant clones or micropropagations, or parts of plants (e.g. harvested tissues or organs), such as plant cuttings, vegetative propagations, embryos, pollen, ovules, flowers, leaves, seeds, clonally propagated plants, roots, stems, root tips, grafts, parts of any of these and the like. Also any developmental stage is included, such as seedlings, cuttings prior or after rooting, mature plants or leaves.

“Harvested plant material” refers herein to plant parts (e.g. a leaf detached from the whole plant) which have been collected for further storage and/or further use.

“Harvested seeds” refers to seeds harvested from a line or variety, e.g. produced after self-fertilization or cross-fertilization and collected.

A plant having “(essentially) all the physiological and morphological characteristics” means a plant having essentially all or all the physiological and morphological characteristics when grown under the same environmental conditions of the plant of NUN 01084 from which it was derived, e.g. the progenitor plant, the parent, the recurrent parent, the plant used for tissue- or cell culture, etc. For example, the plant may have all characteristics mentioned in Table 1. In certain embodiments, the plant having “essentially all the physiological and morphological characteristics” are plants having all the physiological and morphological characteristics, except for certain characteristics, such as one, two or three, mentioned, e.g. the characteristic(s) derived from a converted or introduced gene or trait and/or except for the characteristics which differ in an EDV. So, the plant may have all characteristics mentioned in Table 1, except for one, two or three characteristics of Table 1, in which the plant may thus differ.

A plant having one or more or all “essential physiological and/or morphological characteristics” or one or more “distinguishing characteristics” (such as one, two, three, four or five) refers to a plant having (or retaining) one or more, or all, or retaining all except one, two or three of the distinguishing characteristics mentioned in Table 1 when grown under the same environmental conditions that distinguish NUN 01084 from most similar variety PV0293 such distinguishing characteristics being selected from (but not limited to): tip of seedling cotyledon (e.g., the tip of seedling cotyledon of NUN 01084 is pointed while the tip of seedling cotyledon of PV0293 is round); color of seedling cotyledon of NUN 01084 is darker green than the color of seedling cotyledon of PV0293 (e.g., the RHS Chart Value for the green color of seedling cotyledon of NUN 01084 is 138A while the RHS Chart Value for PV0293 is 144A); margin of first foliage leaves; shape of leaf (prime market stage); base of leaf (prime market stage); tip of leaf (prime market stage); margin of leaf (prime market stage); blade lobing of leaf (prime market stage).

The physiological and/or morphological characteristics mentioned above are commonly evaluated at significance levels of 1%, 5%, 8% or 10% significance level, when measured under the same environmental conditions. For example, a progeny plant of NUN 01084 may have one or more (or all, or all except one, two or three) of the essential physiological and/or morphological characteristics of NUN 01084 listed in Table 1, or one or more or all (or all except one, two or three) of the distinguishing characteristics of NUN 01084 listed in Table 1 and above, as determined at the 1% or 5% significance level when grown under the same environmental conditions.

As used herein, the term “variety” or “cultivar” means a plant grouping within a single botanical taxon of the lowest known rank, which grouping, irrespective of whether the conditions for the grant of a breeder's right are fully met, can be defined by the expression of the characteristics resulting from a given genotype or combination of genotypes, distinguished from any other plant grouping by the expression of at least one of the said characteristics and considered as a unit with regard to its suitability for being propagated unchanged.

A variety is referred to as an “Essentially Derived Variety” (EDV) i.e., shall be deemed to be essentially derived from another variety, “the initial variety” when (i) it is predominantly derived from the initial variety, or from a variety that is itself predominantly derived from the initial variety, while retaining the expression of the essential characteristics that result from the genotype or combination of genotypes of the initial variety; (ii) it is clearly distinguishable from the initial variety; and (iii) except for the differences which result from the act of derivation, it conforms to the initial variety in the expression of the essential characteristics that result from the genotype or combination of genotypes of the initial variety. Thus, an EDV may be obtained for example by the selection of a natural or induced mutant, or of a somaclonal variant, the selection of a variant individual from plants of the initial variety, backcrossing, or transformation by genetic engineering.

“Plant line” is for example a breeding line which can be used to develop one or more varieties.

“Hybrid variety” or “F1 hybrid” refers to the seeds harvested from crossing two inbred (nearly homozygous) parental lines. For example, the female parent is pollinated with pollen of the male parent to produce hybrid (F1) seeds on the female parent.

“Regeneration” refers to the development of a plant from cell culture or tissue culture or vegetative propagation.

“Vegetative propagation”, “vegetative reproduction” or “clonal propagation” are used interchangeably herein and mean the method of taking part of a plant and allowing that plant part to form at least roots where plant part is, e.g., defined as or derived from (e.g. by cutting of) leaf, pollen, embryo, cotyledon, hypocotyl, cells, protoplasts, meristematic cell, root, root tip, pistil, anther, flower, shoot tip, shoot, stem, petiole, etc. When a whole plant is regenerated by vegetative propagation, it is also referred to as a vegetative propagation.

“Selfing” refers to self-pollination of a plant, i.e., the transfer of pollen from the anther to the stigma of the same plant. “Crossing” refers to the mating of two parent plants.

“Average” refers herein to the arithmetic mean.

“Locus” (plural loci) refers to the specific location of a gene or DNA sequence on a chromosome. A locus may confer a specific trait.

“Allele” refers to one or more alternative forms of a gene locus. All of these loci relate to one trait. Sometimes, different alleles can result in different observable phenotypic traits, such as different pigmentation. However, many variations at the genetic level result in little or no observable variation. If a multicellular organism has two sets of chromosomes, i.e. diploid, these chromosomes are referred to as homologous chromosomes. Diploid organisms have one copy of each gene (and therefore one allele) on each chromosome. If both alleles are the same, they are homozygotes. If the alleles are different, they are heterozygotes.

“Genotype” refers to the genetic composition of a cell or organism.

The term “traditional breeding techniques” encompasses herein crossing, selfing, selection, double haploid production, embryo rescue, protoplast fusion, marker assisted selection, mutation breeding etc. as known to the breeder (i.e. methods other than genetic modification/transformation/transgenic methods), by which, for example, a genetically heritable trait can be transferred from one spinach line or variety to another.

“Backcrossing” is a traditional breeding technique used to introduce a trait into a plant line or variety. The plant containing the trait is called the donor plant and the plant into which the trait is transferred is called the recurrent parent. An initial cross is made between the donor parent and the recurrent parent to produce progeny plants. Progeny plants which have the trait are then crossed to the recurrent parent. After several generations of backcrossing and/or selfing the recurrent parent comprises the trait of the donor. The plant generated in this way may be referred to as a “single trait converted plant”.

“Progeny” as used herein refers to plants derived from a plant designated NUN 01084. Progeny may be derived by regeneration of cell culture or tissue culture or parts of a plant designated NUN 01084 or selfing of a plant designated NUN 01084 or by producing seeds of a plant designated NUN 01084. In further embodiments, progeny may also encompass plants derived from crossing of at least one plant designated NUN 01084 with another spinach plant of the same or another variety or (breeding) line, or with a wild spinach plant, backcrossing, inserting of a locus into a plant or selecting a plant comprising a mutation or selecting a variant. A progeny is, e.g., a first generation progeny, i.e. the progeny is directly derived from, obtained from, obtainable from or derivable from the parent plant by, e.g., traditional breeding methods (selfing and/or crossing) or regeneration. However, the term “progeny” generally encompasses further generations such as second, third, fourth, fifth, sixth, seventh or more generations, i.e., generations of plants which are derived from, obtained from, obtainable from or derivable from the former generation by, e.g., traditional breeding methods, regeneration or genetic transformation techniques. For example, a second generation progeny can be produced from a first generation progeny by any of the methods mentioned above. Especially progeny of NUN 01084 which are EDVs or which retain all (or all except 1, 2 or 3) physiological and/or morphological characteristics of NUN 01084 listed in Table 1, or which retain all (or all except 1, 2, or 3) of the distinguishing characteristics of NUN 01084 described elsewhere herein and in Table 1, are encompassed herein.

“Tissue Culture” refers to a composition comprising isolated cells of the same or a different type or a collection of such cells organized into parts of a plant.

The terms “gene converted” or “conversion plant” in this context refer to spinach plants which are developed by backcrossing wherein essentially all of the desired morphological and physiological characteristics of parent are recovered in addition to the one or more genes transferred into the parent via the backcrossing technique or via genetic engineering. Likewise a “Single Locus Converted (Conversion) Plant” refers to plants which are developed by plant breeding techniques comprising or consisting of backcrossing, wherein essentially all of the desired morphological and physiological characteristics of a spinach variety are recovered in addition to the characteristics of the single locus having been transferred into the variety via the backcrossing technique and/or by genetic transformation.

“Transgene” or “chimeric gene” refers to a genetic locus comprising a DNA sequence which has been introduced into the genome of a spinach plant by transformation. A plant comprising a transgene stably integrated into its genome is referred to as “transgenic plant”.

“Haploid” refers to a cell or organism having one set of the two sets of chromosomes in a diploid.

“Diploid” refers to a cell or organism having two sets of chromosomes.

“Triploid” refers to a cell or organism having three sets of chromosomes.

“Tetraploid” refers to a cell or organism having four sets of chromosomes.

The term “mean” refers to the arithmetic mean of several measurements. The skilled person understands that the appearance of a plant depends to some extent on the growing conditions of said plant. Thus, the skilled person will know typical growing conditions for spinach described herein. The mean, if not indicated otherwise within this application, refers to the arithmetic mean of measurements on at least 10 different, randomly selected plants of a variety or line.

“Substantially equivalent” refers to a characteristic that, when compared, does not show a statistically significant difference (e.g., p=0.05) from the mean.

### DETAILED DESCRIPTION OF THE INVENTION

A number of characteristics are important to spinach breeders including (but not limited to) disease resistance, earliness/start of bolting (early, medium, late), growth habit and leaf characteristics.

The present invention relates to a Spinacia oleracea L. hybrid variety, referred to as NUN 01084, or progeny, or EDVs thereof, which has an elliptic shape of first foliage leaves with a flat margin and a lighter lower surface color compared with the upper surface color of said leaves and which has a five sided leaves at prime market stage with a straight base and appointed tip. NUN 01084 is considered most similar to PV 0293, a commercially available variety of Pop Vriend Seeds.

However, NUN 01084 can be easily distinguished from PV 01084. For example, the form of the tip of seedling cotyledons is different, the color of seedling cotyledons of NUN 01084 is darker green than the color of seedling cotyledons of PV 01084, the margin of first foliage leaves of NUN 01084 is different from the margin of first foliage leaves of PV 01084, the shape, base, tip and margin of leaves of NUN 01084 at prime market stage is different from the shape, base, tip and margin of first foliage leaves of PV 01084, the shape, base, tip and margin of leaves of PV 01084 when grown under the same environmental conditions.

Variety NUN 01084 provided herein differs from the most similar comparison variety PV 01084, when grown under the same environmental conditions, in one or more characteristics (referred herein to as “distinguishing characteristics” or “distinguishing morphological and/or physiological characteristics” (or essential physiological and/or morphological characteristics) selected from:
The tip of seedling cotyledon of NUN 01084 is more pointed than the tip of seedling cotyledons of PV0293 (e.g., the tip of seedling cotyledons of NUN 01084 is pointed while the tip of seedling cotyledons of PV0293 is rounded when grown under the same environmental conditions, see USDA descriptors),
the margin of first foliage leaves of NUN 01084 is flat while the margin of first foliage leaves of PV 0293 is slightly curled when grown under the same environmental conditions (see USDA descriptors),
the shape of leaves at prime market stage of NUN 01084 is five-sided while the shape of leaves at prime market stage of PV 0293 is circular when grown under the same environmental conditions (see USDA descriptors),
the base of leaves at prime market stage of NUN 01084 is straight while the base of leaves at prime market stage of PV 0293 is more lobed (see USDA descriptors) when grown under the same environmental conditions (see USDA descriptors),
the tip of leaves at prime market stage of NUN 01084 is pointed while the tip of leaves at prime market stage of PV 0293 is round (see USDA descriptors) when grown under the same environmental conditions,
the margin of leaves at prime market stage of NUN 01084 is flat while the margin of leaves at prime market stage of PV 0293 is curled under (see USDA descriptors) when grown under the same environmental conditions,
the blade of leaves at prime market stage of NUN 01084 is lobed while the blade of leaves at prime market stage of PV 0293 is not lobed (see USDA descriptors) when grown under the same environmental conditions.

It is understood that “significant” differences refer to statistically significant differences, when comparing the characteristic between two plant lines or varieties when grown under the same conditions. Preferably at least about 10, 15, 20, 30, 40, 50 or more plants per line or variety are grown under the same conditions and characteristics are measured on at least about 10, 15, 20 or more randomly selected plant or plant parts to obtain averages. Thus, physiological and morphological characteristics or traits are commonly evaluated at a significance level of 1%, 5%, 8% or 10%, when measured in plants grown under the same environmental conditions. For example, two replications of 50 plants each, from which 20 plants or plant parts were randomly selected can be used to measure characteristics.

In one aspect, the invention provides seeds of the spinach hybrid variety designated NUN 01084 wherein a representative sample of seeds of said variety was deposited under the Budapest Treaty, with Accession number NCIMB ______.

Seeds of NUN 01084 are obtainable by crossing the male parent with the female parent and harvesting the seeds produced on the female parent. The resultant NUN 01084 seeds can be grown to produce NUN 01084 plants. In one embodiment a plurality of NUN 01084 seeds are packaged into small and/or large containers (e.g., bags, cartons, cans, etc.). The seeds may be primed and/or treated with various compounds, such as seed coatings and/or fungicides and/or insecticides.

Also provided are plants of spinach hybrid variety NUN 01084 produced from seeds or vegetative propagation, wherein a representative sample of said seeds has been deposited under the Budapest Treaty, with Accession Number NCIMB ______. For example, plants of NUN 01084 can be produced by seeding directly in the ground (e.g., field) or by germinating the seeds in controlled environment conditions (e.g., greenhouses) and then transplanting the seedlings into the field.

In accordance with one aspect of the present invention, there is provided a plant having all the physiological and morphological characteristics of NUN 01084. A description of the physiological and morphological characteristics of spinach variety NUN 01084 is presented, e.g., in Table 1.

In other aspects, the invention provides for progeny of variety NUN 01084 such as progeny obtained by further breeding with NUN 01084. Further breeding with NUN 01084 includes selfing NUN 01084 one or more times and/or cross-pollinating NUN 01084 with another spinach plant or variety one or more times.

In particular, the invention provides for progeny that retain all or all except one, two or three of the essential morphological and physiological characteristics of NUN 01084 or that retain one or more or all or all except one, two or three of the distinguishing characteristics of the spinach type described further above and when grown under the same environmental conditions as NUN 01084.

In another aspect, the invention provides for vegetative reproductions of the variety NUN 01084, or of progeny thereof, and the invention also provided for essentially derived varieties (EDVs) of NUN 01084, or of progeny thereof, and vegetative reproductions of such EDVs.

The morphological and/or physiological differences between plants according to the invention, i.e. NUN 01084 or progeny thereof, or an EDV thereof, and other known varieties can easily be established by growing NUN 01084 (or progeny or EDVs) next to the other varieties (in the same field, under the same environmental conditions), preferably in several locations which are suitable for said spinach cultivation, and measuring morphological and/or physiological characteristics of a number of plants (e.g., to calculate an average value and to determine the variation range/uniformity within the variety). For example, trials can be carried out in Acampo Calif., USA (N 38 degrees 07′261″/W 121 degrees 18′ 807″, USA), whereby e.g., ploidy; days from planting to prime market stage; plant habit; plant size; width, length, tip and color of seedling cotyledons; shape, base, tip, margin, upper and lower surface color of first foliage leaves; surface, shape, base, tip, margin, upper and lower surface color, luster, blade size, blade lobing, petiole colorpetiole red pigmentation, petiole length and petiole diameter of leaves at prime market stage; seed stalk development; seed surface; disease resistances and/or insect resistances can be measured and directly compared (see USDA descriptors).

Morphological and/or physiological characteristics (and distinguishing characteristics which distinguish NUN 01084 from PV 0293) of NUN 01084, are provided in the Examples, in Table 1. Encompassed herein are also plants derivable from NUN 01084 (e.g. by selfings and/or crossing and/or backcrossing with NUN 01084 and/or mutant selection and/or natural variant selection) comprising all (or all except one, two or three) of the physiological and/or morphological characteristics of NUN 01307 listed in Table 1 as determined at the 5% significance level when grown under the same environmental conditions and/or comprising one or more (or all; or all except one, two or three) of the distinguishing characteristics as determined at the 5% significance level when grown under the same environmental conditions. The skilled person understands that the morphological and/or physiological characteristics of a plant may vary somewhat with variation in the environment (such as temperature, light intensity, day length, humidity, soil, fertilizer use), which is why a comparison under the same environmental conditions is preferred. Colors can best be measured against The Munsell Book of Color (Munsell Color Macbeth Division of Kollmorgan Instruments Corporation) or using the Royal Horticultural Society Chart (http://www.rhs.org.uk/Plants/RHS-Publications/RHS-colour-charts).

Also at-harvest and/or post-harvest characteristics of leaves can be compared. For example, cold storage holding quality (e.g., yellowing) can be measured using known methods.

An EDV according to the invention is an Essentially Derived Variety of NUN 01084 having one, two or three physiological and/or morphological characteristics which are different from those of NUN 01084 and which otherwise has all the physiological and morphological characteristics of NUN 01084, wherein a representative sample of seed of variety NUN 01084 has been deposited under Accession Number NCIMB ______. In particular, variants which differ from NUN 01084 in one, two or three of the characteristics mentioned in Table 1 are encompassed.

In one aspect, the EDV differs from NUN 01084 in one, two or three of the distinguishing morphological and/or physiological characteristics selected from: i) upper and/or lower leaf surface color of first foliage leaf or of leaf at prime market stage, ii) leaf shape of leaf at prime market stage, iii) leaf base of leaf at prime market stage, iv) blade lobing of leaf at prime market stage, v) petiole length of leaf at prime market stage.

In another embodiment the EDV may differ from NUN 01084 in one, two or three morphological and/or physiological characteristic other than the “distinguishing morphological and/or physiological characteristics” (or essential physiological and/or morphological characteristics) of NUN 01084 for example selected from (but not limited to): i) ploidity, ii) plant habit at prime market stage, iii) shape of first foliage leaf, iv) base of first foliage leaf, v) tip of first foliage leaf, vi) surface of leaf at prime market stagepetiole red pigmentation of leaf at prime market stage, vii) seed surface or other characteristics.

In still yet another aspect of the invention, the genetic complement of NUN 01084 is provided. The phrase “genetic complement” is used to refer to the aggregate of nucleotide sequences, the expression of which sequences defines the phenotype of, in the present case, a spinach plant, or a cell or tissue of that plant. A genetic complement thus represents the genetic make-up of a cell, tissue or plant, and a hybrid genetic complement represents the genetic make-up of a hybrid cell, tissue or plant.

The invention thus provides spinach plant cells that have a genetic complement in accordance with the spinach plant cells disclosed herein, and plants, seeds and plants containing such cells. Plant genetic complements may be assessed by genetic marker profiles, and by the expression of phenotypic traits that are characteristic of the expression of the genetic complement, e.g., isozyme typing profiles. It is understood that NUN 01084 could be identified by any of the many well-known techniques such as, for example, Simple Sequence Length Polymorphisms (SSLPs), Randomly Amplified Polymorphic DNAs (RAPDs), DNA Amplification Fingerprinting (DAF), Sequence Characterized Amplified Regions (SCARs), Arbitrary Primed Polymerase Chain Reaction (AP-PCR), Amplified Fragment Length Polymorphisms (AFLPs) (see, e.g., EP 534 858), and Single Nucleotide Polymorphisms (SNPs).

In other aspects, the invention provides for a plant part of NUN 01084 or of a progeny of NUN 01084 (such as a plant having all the physiological and morphological characteristics of NUN 01084 or a hybrid of NUN 01084), or a derived variety of NUN 01084, e.g., an EDV, (such as a plant having all except one, two or three of the morphological and physiological characteristics of NUN 01084). Parts of NUN 01084 (or from its progeny or from an EDV of NUN 01084) encompass any cells, tissues, organs obtainable from the seedlings or plants, such as but not limited to: spinach leaves or parts thereof, cuttings, hypocotyl, cotyledon, pollen, flowers, anthers, embryos, ovaries, and the like. Preferably, a part of a plant part of NUN 01084 or its progeny or an EDV of NUN 01084 is a leaf, pollen, flowers, shoots or cuttings or parts thereof. Such parts can be stored and/or processed further. Encompassed are therefore also food or feed products comprising one or more of such parts, such as fresh, dried, frozen spinach leaves or parts thereof, etc. from NUN 01084 or from progeny thereof, or from a derived variety, such as an EDV. In another embodiment, the invention provides for a container comprising or consisting of a plurality of harvested spinach leaves of NUN 01084, or progeny thereof, or a derived variety, such as an EDV. For example, containers such as cans, boxes, crates, bags, cartons, Modified Atmosphere Packagings, films (e.g. biodegradable films), etc. comprising plant parts of plants (fresh and/or processed) described herein are also provided herein.

The invention also provides a spinach plant comprising at least a first set of the chromosomes of spinach variety NUN 01084, a sample of seed having been deposited under Accession Number NCIMB ______; further provided is a spinach plant comprising a single locus conversion, wherein said spinach plant has essentially all of the morphological and/or physiological characteristics of the spinach plant comprising at least a first set of the chromosomes of spinach variety NUN 01084. In another embodiment, this single locus conversion confers a trait selected from the group consisting of male sterility, herbicide tolerance, insect resistance, pest resistance, disease resistance, environmental stress tolerance, modified carbohydrate metabolism and modified protein metabolism.

In one embodiment, NUN 01084 may also be mutated (by e.g. irradiation, chemical mutagenesis, heat treatment, etc.) and mutated seeds or plants may be selected in order to change one or more characteristics of NUN 01084, e.g. one, two, three or more of the essential physiological and/or morphological characteristics of NUN 01084 may be changed. The one, two, three or more changed characteristics may be one, two or three of the distinguishing characteristics of NUN 01084. Also natural mutants or natural variants of NUN 01084 may be identified and used in breeding. Methods such as TILLING and/or EcoTILLING may be applied to spinach populations in order to identify mutants. Similarly, NUN 01084 may be transformed and regenerated, whereby one or more chimeric genes are introduced into the variety or progeny thereof or into an EDV thereof. Transformation can be carried out using standard methods, such as Agrobacterium tumefaciens mediated transformation or biolistics, followed by selection of the transformed cells and regeneration into plants. A desired trait (e.g. genes conferring pest or disease resistance, herbicide, fungicide or insecticide tolerance, etc.) can be introduced into NUN 01084, or progeny thereof, by transforming NUN 01084 or progeny thereof with a transgene that confers the desired trait, wherein the transformed plant retains all the phenotypic and/or morphological and/or physiological characteristics of NUN 01084 or the progeny thereof and contains the desired trait. Thus, also a transgenic spinach plant comprising a transgene conferring a desired trait and further comprising all (or all except one two or three) of the morphological and/or physiological characteristics of NUN 01084, or further comprising all (or all except one, two or three) of the distinguishing characteristics of NUN 01084, is provided herein.

In one aspect, haploid plants and/or double haploid plants of NUN 01084, or an EDV of NUN 01084, or progeny of any of these, are encompassed herein. Haploid and double haploid (DH) plants can, for example, be produced by cell or tissue culture and chromosome doubling agents and regeneration into a whole plant. For DH production chromosome doubling may be induced using known methods, such as colchicine treatment or the like.

The invention also provides for a method of producing a new spinach plant. The method comprises crossing a plant of the invention NUN 01084, either as male or as female parent, with a second spinach plant (or a wild relative of spinach) one or more times, and/or selfing a spinach plant NUN 01084 one or more times and optionally selecting progeny from said crossing and/or selfing. In one embodiment, the said second spinach plant is a Spinacia oleracea L plant. Thus, invention also provides a method for developing a spinach plant in a spinach breeding program, using a spinach plant of the invention or a part thereof as a source of plant breeding material. Suitable plant breeding techniques are recurrent selection, backcrossing, pedigree breeding, mass selection, mutation breeding and/or genetic marker enhanced selection.

In one embodiment, progeny of a plant according to the invention are either the generation (seeds) produced from the first cross (F1) or selfing (51), or any further generation produced by crossing and/or selfing (F2, F3, etc.) and/or backcrossing (BC1, BC2, etc.) one or more selected plants of the F1 and/or S1 and/or BC1 generation (or plants of any further generation, e.g. the F2) with another spinach plant (and/or with a wild relative of spinach). Progeny may have all (or all except one, two or three) of the physiological and/or morphological characteristics of spinach variety NUN 01084 when grown under the same environmental conditions and/or progeny may have (be selected for having) one or more, or all or all except one, two or three of the distinguishing characteristics of spinach of the invention. Using common breeding methods such as backcrossing or recurrent selection, one or more specific characteristics may be introduced into NUN 01084, to provide an EDV of NUN 01084.

One embodiment of the present invention refers to spinach seeds and plants produced by a process that comprises crossing a first parent spinach plant with a second parent spinach plant, wherein at least one of the first or second parent spinach plants is a plant of the variety designated NUN 01084. In one preferred embodiment of the invention, spinach seed and plants produced by the process are first generation (F1) hybrid spinach seed and plants produced by crossing a plant in accordance with the invention with another, distinct plant. The present invention further contemplates plant parts of such an F1 hybrid spinach plant, and methods of use thereof.

The development of new varieties using one or more starting varieties is well known in the art. In accordance with the present invention, processes are provided for producing spinach seeds, plants and fruit, which processes generally comprise crossing a first parent spinach plant with a second parent spinach plant, wherein at least one of the first or second parent spinach plants is a plant of the variety designated NUN 01084. Thus, in accordance with the invention, novel varieties may be created by crossing NUN 01084 optionally followed by multiple generations of breeding according to such well known methods. These processes may be further exemplified as processes for preparing hybrid spinach seed or plants comprising crossing a plant designated NUN 01084 with a second spinach plant one or more times, and selecting progeny from said crossing. In these processes, crossing will result in the production of seed. The seed production occurs regardless of whether the seed is collected or not.

New varieties may be created by crossing with any second plant. In selecting such a second plant to cross for the purpose of developing novel lines, it may be desired to choose those plants which either themselves exhibit one or more selected desirable characteristics or which exhibit the desired characteristic(s) in progeny. Once initial crosses have been made, inbreeding and selection take place to produce new varieties. For development of a uniform line, often five or more generations of selfing and selection are involved.

In one embodiment of the invention, the first step in “crossing” comprises planting seeds of a first and second parent spinach plant, often in proximity so that pollination will occur for example, mediated by insect vectors. Alternatively, pollen can be transferred manually. Where the plant is self-pollinated, pollination may occur without the need for direct human intervention other than plant cultivation.

A second step may comprise cultivating or growing the seeds of first and second parent spinach plants into plants that bear flowers. A third step may comprise preventing self-pollination of the plants, such as by emasculating the male portions of flowers, (i.e., treating or manipulating the flowers to produce an emasculated parent spinach plant). Self-incompatibility systems may also be used in some hybrid crops for the same purpose. Self-incompatible plants still shed viable pollen and can pollinate plants of other varieties but are incapable of pollinating themselves or other plants of the same line.

A fourth step for a hybrid cross may comprise cross-pollination between the first and second parent spinach plants. Yet another step comprises harvesting the seeds from at least one of the parent spinach plants. The harvested seed can be grown to produce a spinach plant or hybrid spinach plant.

Backcrossing can also be used to improve an inbred plant. Backcrossing transfers a specific desirable trait from one inbred or non-inbred source to an inbred that lacks that trait.

In a typical backcross protocol, the original variety of interest (recurrent parent) is crossed to a second variety (non-recurrent parent) that carries the single locus of interest to be transferred. The resulting progeny from this cross are then crossed again to the recurrent parent and the process is repeated until a spinach plant is obtained wherein essentially all of the desired morphological and physiological characteristics of the recurrent parent are recovered in the converted plant, in addition to the single transferred locus from the non-recurrent parent. The selection of a suitable recurrent parent is an important step for a successful backcrossing procedure. The goal of a backcross protocol is to alter or substitute a single trait or characteristic in the original variety. To accomplish this, a single locus of the recurrent variety is modified or substituted with the desired locus from the non-recurrent parent, while retaining essentially all of the rest of the desired genetic, and therefore the desired physiological and morphological constitution of the original variety. The choice of the particular non-recurrent parent will depend on the purpose of the backcross; one of the major purposes is to add some commercially desirable trait to the plant. The exact backcrossing protocol will depend on the characteristic or trait being altered to determine an appropriate testing protocol. Although backcrossing methods are simplified when the characteristic being transferred is a dominant allele, a recessive allele may also be transferred. In this instance it may be necessary to introduce a test of the progeny to determine if the desired characteristic has been successfully transferred.

In one embodiment, progeny diploid spinach plants of a backcross in which NUN 01084 is the recurrent parent comprise (i) the desired trait from the non-recurrent parent and (ii) all of the physiological and morphological characteristics of NUN 01084 as determined at the 5% significance level when grown in the same environmental conditions.

Spinach varieties can also be developed from more than two parents. The technique, known as modified backcrossing, uses different recurrent parents during the backcrossing. Modified backcrossing may be used to replace the original recurrent parent with a variety having certain more desirable characteristics or multiple parents may be used to obtain different desirable characteristics from each. Many single locus traits have been identified that are not regularly selected for in the development of a new inbred but that can be improved by backcrossing techniques. Single locus traits mayor may not be transgenic; examples of these traits include, but are not limited to, male sterility, herbicide resistance, resistance to bacterial, fungal, or viral disease, insect resistance, restoration of male fertility, modified fatty acid or carbohydrate metabolism, and enhanced nutritional quality. These comprise genes generally inherited through the nucleus.

Examples of desirable characteristics may include, but are not limited to, resistance to one or more of the following diseases Peronosporafarinose f.sp. spinaciae, e.g. to new races and/or race 510C; white rust (Albugo occidentalis), Fusarium oxysporum f.sp. spinaciae, Pythium resistance, Rhizoctonia resistance, Colletotrichum anthracnose resistance, Cercospora beticola resistance, Verticillium dahliae resistance, Phytophthora ssp resistance, Stemphylium leaf spot resistance, Curly Top Virus resistance, Cucumber Mosaic Virus (CMV) resistance, Impatiens Necrotic Spot Virus (INSV), Beet Yellows and/or Beet mosaic resistance, leaf miner resistance, and others.

Non-limiting examples of genes that may be utilized for generating transgenic spinach include RAR1 disease resistance proteins (see, e.g., U.S. Pat. No. 7,098,378, the ability to tolerate high salt conditions, as described in (see, e.g., U.S. Pat. No. 7,041,875); trehalose synthase for increased amounts of trehalose to increase tolerance to a variety of stresses, in particular to decreased availability of water (see, e.g., U.S. Pat. No. 5,792,92); overexpression of phytochrome, such as for increased shade tolerance and/or darker green color, as described in, for example, U.S. Pat. No. 5,268,526; expression of reversibly glycosylated protein (RGP) for at least altered growth rates, as described in, for example, U.S. Pat. No. 6,194,638; improved growth under low-light conditions (see, e.g., U.S. Pat. No. 7,081,363).

One aspect of the present invention refers to a method of introducing a single locus conversion, or single trait conversion, into a spinach plant according to the invention and/or into NUN 01084 comprising:

(a) crossing a spinach plant of variety NUN 01084, a representative sample of seed of said variety having been deposited under Accession Number NCIMB ______, with a second spinach plant comprising a desired single locus to produce F1 progeny plants;
(b) selecting F1 progeny plants that have the single locus to produce selected F1 progeny plants;
(c) crossing the selected progeny plants with a plant of NUN 01084, to produce backcross progeny plants;
(d) selecting backcross progeny plants that have the single locus and one or more or all (or all except one, two or three) of the distinguishing characteristics of spinachs according to the invention and/or all (or all except one, two or three) of the physiological and/or morphological characteristics of NUN 01084 to produce selected backcross progeny plants; and
(e) repeating steps (c) and (d) one or more times in succession to produce selected second, third, fourth or higher backcross progeny plants that comprise the single locus and otherwise one or more or all distinguishing characteristics of the spinachs according to the invention and/or comprise all of the physiological and morphological characteristics of NUN 01084, when grown in the same environmental conditions.

The invention provides also for methods of producing EDVs (Essentially Derived Varieties) of NUN 01084 which differ from NUN 01084 in one, two, three or more morphological and/or physiological characteristics, but which are still genetically closely related to NUN 01084. The relatedness can, for example be determined by fingerprinting techniques (e.g., making use of isozyme markers and/or molecular markers such as SNP markers, AFLP markers, microsatellites, SSR markers, minisatellites, RAPD markers, RFLP markers and others). A plant is “closely related” to NUN 01084 if its DNA fingerprint is at least 80%, 90%, 95% or 98% identical to the fingerprint of NUN 01084. In one embodiment AFLP markers are used for DNA fingerprinting (Vos et al. 1995) or other markers, such as microsatellite markers. A closely related plant may have a Jaccard's Similarity index of at least about 0.8, preferably at least about 0.9, 0.95, 0.95, 0.96, 0.97, 0.98 or more (Staub et al 2000). The invention also provides plants and varieties obtained by these methods. In one embodiment, relatedness and whether a variety is an EDV of NUN 01084, may be determined according to the Variety Tracer method of the Nak Tuinbouw (http://www.naktuinbouw.nl/en/topic/identification-using-dna).

In yet another aspect the invention provides a method of producing a spinach plant, comprising selfing a plant designated NUN 01084 one or more times, and selecting progeny from said selfing.

Spinach according to the invention, such as the variety NUN 01084, or its progeny or an EDV of NUN 01084, can also be reproduced using vegetative reproduction methods. Therefore, the invention provides for a method of producing plants, or a part thereof, of variety NUN 01084, or of progeny thereof, or of an EDV of NUN 01084, comprising vegetative propagation of variety NUN 01084 or of progeny thereof, or of an EDV of NUN 01084. Vegetative propagation comprises regenerating a whole plant from a part of variety NUN 01084 (or from its progeny or from an EDV of NUN 01084), such as a cutting, a cell culture or a tissue culture.

Accordingly, the invention also provides for a vegetatively propagated plant of variety NUN 01084 (or from its progeny or from an EDV of NUN 01084), or a part thereof, having one, two, three or more distinguishing characteristics or all the morphological and physiological characteristics of NUN 01084 (except for the characteristics differing in the EDV), when grown under the same environmental conditions.

Also included is a cell culture or tissue culture produced from a plant designated NUN 01084. The tissue culture will preferably be capable of regenerating plants capable of expressing all of the physiological and morphological characteristics of NUN 01084 or all except one, two or three of the morphological and physiological characteristics of NUN 01084. The regenerable cells in such tissue cultures may be derived, for example, from embryos, meristems, cotyledons, pollen, leaves, anthers, roots, root tips, pistil, flower, seed and stalks. Still further, the present invention provides spinach plants regenerated from a tissue culture of the invention, the plants having all the physiological and morphological characteristics of NUN 01084 or all except one, two or three of the morphological and physiological characteristics of NUN 01084.

All documents (e.g., patent publications) are herein incorporated by reference in their entirety.

### Examples

### Development of NUN 01084

The hybrid NUN 01084 was developed from a male and female proprietary inbred line of Nunhems. The female and male parents were crossed to produce hybrid (F1) seeds of NUN 01084. The seeds of NUN 01084 can be grown to produce hybrid plants and parts thereof (e.g. spinach leaves). The hybrid NUN 01084 can be propagated by seeds or vegetative.

The hybrid variety is uniform and genetically stable. This has been established through evaluation of horticultural characteristics. Several hybrid seed production events resulted in no observable deviation in genetic stability. Coupled with the confirmation of genetic stability of the female and male parents the Applicant concluded that NUN 01084 is uniform and stable.

### Deposit Information

A total of 2500 seeds of the hybrid variety NUN 01084 were deposited according to the Budapest Treaty by Nunhems B.V. on ______, at the American Type Culture Collection (ATCC), University Boulevard, Manassas, Va. 20110-2209 USA or at the NCIMB Ltd., Ferguson Building, Craibstone Estate, Bucksburn, Aberdeen AB21 9YA, United Kingdom (NCIMB). The deposit has been assigned Accession Number PTA ______ or NCIMB ______.

A deposit of NUN 01084 and of the male and female parent line is also maintained at Nunhems B.V. Access to the deposit will be available during the pendency of this application to persons determined by the Director of the U.S. Patent Office to be entitled thereto upon request. Subject to 37 C.F.R. §1.808(b), all restrictions imposed by the depositor on the availability to the public of the deposited material will be irrevocably removed upon the granting of the patent. The deposit will be maintained for a period of 30 years, or 5 years after the most recent request, or for the enforceable life of the patent whichever is longer, and will be replaced if it ever becomes nonviable during that period. Applicant does not waive any rights granted under this patent on this application or under the Plant Variety Protection Act (7 USC 2321 et seq.).

Most similar variety to NUN 01084 is PV 0293, a commercial variety from Pop Vriend Seeds. In Table 1 a comparison between NUN 01084 and most similar variety PV 0293 is shown based on a trial in the USA. Trial location: Acampo Calif. USA, (coordinates: 38°07′261″N, −121°18′80547″W), USA 2011/2012. Planting date: seeding date Nov. 6, 2011, transplanting date Nov. 4, 2011 and harvested Mar. 13, 2012.

Two replications of 50 plants each, from which 20 plants or plant parts were randomly selected to measure characteristics. In Table 1 the USDA descriptors of NUN 01084 (this application) and reference Pluston (commercial variety) and Poulton (commercial variety) are summarized.

TABLE 1
Comparison between NUN 01084 and variety PV 0293
which is considered the most similar variety.

| | NUN | PV |
|---|---|---|
| CHARACTERISTIC | 01084 | 0293 |
| Species | Spinacia | Spinacia |
| | oleracea | oleracea |
| | L. | L. |
| PLOIDY | | |
| 1 = Diploid; | | |
| 2 = tetraploid; | | |
| 3 = other | | |
| MATURITY | | |
| Growth Rate | | |
| 1 = slow; | | |
| 2 = medium (Long Standing Bloomsdale); | | |
| 3 = fast (Dixie Market) | | |
| Days from planting to prime market stage | | |
| PLANT (Prime Market Stage): | | |
| Habit | 1 | 1 |
| 1 = flat (Viroflay); | | |
| 2 = semi-erect (Long Standing Bloomsdale) | | |
| 3 = erect (Virginia Savoy) | | |
| Size | | |
| 1 = small (America); | | |
| 2 = medium; | | |
| 3 = large (Giant Nobel) | | |
| Spread (cm) | | |
| Height (cm) | | |
| SEEDLING COTYLEDON | | |
| Width (mm) | 3.62 mm | 5.60 mm |
| Length (mm) | 46.55 mm | 45.35 mm |
| Tip | 1 | 2 |
| 1 = pointed; | | |
| 2 = rounded | | |
| Color | 3 | 2 |
| 1 = light green; | | |
| 2 = medium green; | | |
| 3 = dark green; | | |
| 4 = other | | |
| Color Chart | RHS | RHS |
| Color Chart Value | 138A | 144A |
| LEAF (First Foliage Leaves) | | |
| Shape | 1 | 1 |
| 1 = elliptic | | |
| 2 = circular; | | |
| 3 = ovate; | | |
| 4 = other | | |
| Base | 1 | 1 |
| 1 = V-base; | | |
| 2 = straight; | | |
| 3 = lobed | | |
| Tip | 1 | 1 |
| 1 = round; | | |
| 2 = round-pointed; | | |
| 3 = pointed | | |
| Margin | 1 | 2 |
| 1 = flat; | | |
| 2 = slightly curled; | | |
| 3 = curled under | | |
| Upper Surface Color | 2 | 2 |
| 1 = light green (Hollandia); | | |
| 2 = medium green (Giant Nobel); | | |
| 3 = dark green (Long Standing Bloomsdale) | | |
| Color Chart | RHS | RHS |
| Color Chart Value | 137A | 141A |
| Lower Surface Color | 1 | 2 |
| 1 = lighter; | | |
| 2 = same; | | |
| 3 = darker | | |
| Color Chart | RHS | RHS |
| Color Chart Value | 137C | 143A |
| LEAF (Prime Market Stage) | | |
| Surface | 2 | 2 |
| 1 = smooth (Viroflay); | | |
| 2 = semi-savoy (Northland); | | |
| 3 = savoy (Virginia Savoy) | | |
| Shape | 5 | 2 |
| 1 = elliptic | | |
| 2 = circular; | | |
| 3 = ovate; | | |
| 4 = three sided; | | |
| 5 = five-sided; | | |
| 6 = arrow-shaped; | | |
| 7 = asymmetrical | | |
| Base | 2 | 3 |
| 1 = V-base; | | |
| 2 = straight; | | |
| 3 = lobed | | |
| Tip | 3 | 1 |
| 1 = round; | | |
| 2 = round-pointed; | | |
| 3 = pointed | | |
| Margin | 1 | 3 |
| 1 = flat; | | |
| 2 = slightly curled; | | |
| 3 = curled under; | | |
| 4 = curled up | | |
| Upper Surface Color | 3 | 2 |
| 1 = light green (Hollandia); | | |
| 2 = medium green (Giant Nobel); | | |
| 3 = dark green (Long Standing Bloomsdale) | | |
| 4 = dull green (Northland) | | |
| Color Chart | — | — |
| Color Chart Value | — | — |
| Lower Surface Color | — | — |
| 1 = lighter; | | |
| 2 = same; | | |
| 3 = darker | | |
| Color Chart | — | — |
| Color Chart Value | — | — |
| Luster | — | — |
| 1 = glossy | | |
| 2 = dull | | |
| Blade Size | — | — |
| 1 = small (Long Standing Bloomsdale); | | |
| 2 = medium (Virginia Savoy); | | |
| 3 = large (Giant Nobel) | | |
| Blade Lobing | 2 | 1 |
| 1 = not lobed; | | |
| 2 = lobed | | |
| Petiole Color | 1 | 1 |
| 1 = white; | | |
| 2 = light yellow; | | |
| 3 = light green; | | |
| 4 = medium green | | |
| Color Chart | — | — |
| Color Chart Value | — | — |
| Petiole Red Pigmentation | 2 | 2 |
| 1 = present; | | |
| 2 = absent | | |
| Petiole Length to the Blade (mm) | — | — |
| Petiol Length | 2 | 1 |
| 1 = short; | | |
| 2 = medium; | | |
| 3 = long (Viroflay) | | |
| Petiole Diameter (mm) | — | — |
| Petiole Diameter | — | — |
| 1 = small; | | |
| 2 = medium; | | |
| 3 = large (Giant Nobel) | | |
| SEED STALK DEVELOPMENT | | |
| Start of Bolting (10% of plants): | — | — |
| 1 = Early (Dixie Market); | | |
| 2 = Medium (Long Standing Bloomsdale); | | |
| 3 = Late (Norgreen) | | |
| Height of Stalk (cm) | — | — |
| Leaves on Stalk of Female Plant: | — | — |
| 1 = Few or None | | |
| 2 = Many | | |
| Leaves on Stalk of Male Plant: | — | — |
| 1 = Few or None | | |
| 2 = Many | | |
| Plants that are Female: | — | — |
| 1 = 0-10% | | |
| 2 = 11-35% | | |
| 3 = 36-65% | | |
| 4 = 66-90% | | |
| 5 = 91-100% | | |
| Plants that are Male: | — | — |
| 1 = 0-10% | | |
| 2 = 11-35% | | |
| 3 = 36-65% | | |
| 4 = 66-90% | | |
| 5 = 91-100% | | |
| Plants that are Monoecious: 1 = 0-10% | — | — |
| 2 = 11-35% 3 = 36-65% 4 = 66-90% | | |
| 5 = 91-100% | | |
| SEED: | | |
| Surface: | 1 | 1 |
| 1 = Smooth; | | |
| 2 = Prickly | | |
| DISEASE REACTION: (0 = Not Tested, | 0 | 0 |
| 1 = Susceptible, 2 = Resistant) | | |
| Downy Mildew (Peronospora spinaciae) Race 1 | 0 | 0 |
| Downy Mildew (Peronospora spinaciae) Race 2 | 0 | 0 |
| Downy Mildew (Peronospora spinaciae) Race 3 | 0 | 0 |
| Fusarium Wilt (Fusarium oxysporum f. sp. | 0 | 0 |
| spinaciae) | | |
| White Rust (Albugo Occidentals) | 0 | 0 |
| Curly Top Virus | 0 | 0 |
| Cucumber Mosaic Virus | 0 | 0 |
| Other (Specify) | 0 | 0 |
| WINTER HARDINESS: | | |
| Hardiness: | | |
| 1 = Not Hardy | | |
| 2 = Moderate | | |
| 3 = Hardy | | |

These are typical values. Values may vary due to environment. Other values that are substantially equivalent are also within the scope of the invention.

### REFERENCES

The following references, to the extent that they provide exemplary procedural or other details supplementary to those set forth herein, are specifically incorporated herein by reference:
http://www.ams.usda.gov/AMSv1.0/getfile?dDocName=STELDEV3003749.
http://www.upov.int/edocs/tgdocs/en/tg055.pdf.
http://www.rhs.org.uk/Plants/RHS-Publications/RHS-colour-charts.
U.S. Pat. No. 5,268,526.
U.S. Pat. No. 5,792,921.
U.S. Pat. No. 6,194,638.
U.S. Pat. No. 7,041,875.
U.S. Pat. No. 7,081,363.
U.S. Pat. No. 7,098,378.
Staub et al 2000, Euphytica 115: 225-241.
Vos et al. 1995, Nucleic Acid Research 23: 4407-4414.
http://www.naktuinbouw.nl/en/topic/identification-using-dna.

1. A seed of NUN 01084 a sample of seed of said variety having been deposited under ATCC Accession Number ______.

2. A plant grown from the seed of claim 1.

3. A plant part of the plant of claim 2.

4. The plant part of claim 3, wherein said part is selected from the group consisting of a seed, pollen, an ovule, a leaf, and a cell.

5. A spinach plant, or a part thereof, having all the physiological and morphological characteristics of the spinach plant of claim 2.

6. A tissue culture of regenerable cells of spinach hybrid variety NUN 01084, a sample of seed of said hybrid variety having been deposited under ATCC Accession Number ______.

7. The tissue culture according to claim 6, comprising cells or protoplasts from a plant part selected from the group consisting of embryos, meristems, cotyledons, pollen, leaves, anthers, roots, root tips, shoots, pistil, flower, seed and stalks.

8. A spinach plant regenerated from the tissue culture of claim 6, wherein the regenerated plant expresses all of the physiological and morphological characteristics of spinach hybrid variety NUN 01084, a sample of seed of said hybrid variety having been deposited under ATCC Accession Number ______.

9. A method of producing seed, comprising crossing the plant of claim 2 with itself or a second spinach plant and allowing seed to form.

10. The method of claim 9, comprising crossing the plant of spinach hybrid variety NUN 01084 with a spinach plant of a different genotype relative to NUN 01084.

11. An F1 hybrid seed produced by the method of claim 10.

12. An F1 hybrid plant produced by growing the seed of claim 11.

13. A method for producing a seed of a NUN 01084-derived spinach plant comprising the steps of:
(a) crossing a spinach plant of hybrid variety NUN 01084, a sample of seed of said variety having been deposited under ATCC Accession Number ______, with a second spinach plant; and
(b) allowing seed of a NUN 01084-derived spinach plant to form.

14. The method of claim 13, further comprising the steps of:
(c) crossing a plant grown from said NUN 01084-derived spinach seed with itself or a second spinach plant to yield additional NUN 01084-derived spinach seed;
(d) growing said additional NUN 01084-derived spinach seed of step (c) to yield additional NUN 01084-derived spinach plants; and
(e) repeating the crossing and growing steps of (c) and (d) to generate further NUN 01084-derived spinach plants.

15. A method of vegetatively propagating a plant of spinach variety NUN 01084 comprising the steps of:
(a) collecting tissue capable of being propagated from a plant of spinach variety NUN 01084, a sample of seed of said variety having been deposited under ATCC Accession Number ______;
(b) cultivating said tissue to obtain proliferated shoots; and
(c) rooting said proliferated shoots to obtain rooted plantlets.

16. The method of claim 15, further comprising growing plants from said rooted plantlets.

17. A method of introducing a desired trait into spinach variety NUN 01084 comprising:
(a) crossing a plant of variety NUN 01084, a sample of seed of said variety having been deposited under ATCC Accession Number ______, with a second spinach plant that comprises a desired trait to produce F1 progeny;
(b) selecting an F1 progeny that comprises the desired trait;
(c) crossing the selected F1 progeny with a plant of variety NUN 01084 to produce backcross progeny;
(d) selecting backcross progeny comprising the desired trait and the physiological and morphological characteristic of spinach variety NUN 01084; and
(e) repeating steps (c) and (d) three or more times in succession to produce selected fourth or higher backcross progeny that comprise the desired trait.

18. A spinach plant produced by the method of claim 17, wherein the spinach plant comprises essentially all of the physiological and morphological characteristics of spinach variety NUN 01084, a sample of seed of said variety having been deposited under ATCC Accession Number ______.

19. A method of producing a plant of spinach variety NUN 01084, a sample of seed of said variety having been deposited under ATCC Accession Number ______, comprising an added desired trait, the method comprising introducing a transgene conferring the desired trait into a plant of spinach variety NUN 01084 to produce of spinach variety NUN 01084 comprising an added desired trait.

20. A progeny plant of the plant of claim 2 that comprises all of the physiological and morphological characteristics of spinach variety NUN 01084, a sample of seed of said variety having been deposited under ATCC Accession Number ______.

21. A seed that produces the plant of claim 20.

22. An Essentially Derived Variety of NUN 01084 having one, two or three physiological and/or morphological characteristics which are different from those of NUN 01084 and which otherwise has all the physiological and morphological characteristics of NUN 01084, wherein a representative sample of seed of variety NUN 01084 has been deposited under Accession Number NCIMB ______.

23. A method of determining the genotype of the plant of claim 2, comprising obtaining a sample of nucleic acids from said plant and detecting in said nucleic acids a plurality of polymorphisms.

24. The method of claim 23, further comprising the step of storing the results of the step of detecting the plurality of polymorphisms on a computer readable medium.

25. A method of producing spinach comprising:
(a) obtaining the plant of claim 2, and
(b) collecting leaf tissue from the plant.
